(19) United States
(12) Patent Application Publication
(10) Pub. No.: US 2003/0212263 A1
(43) Pub. Date: Nov. 13, 2003

(54) Mammastatin sequence variant C

(75) Inventor: Paul R. Ervin JR., Ann Arbor, MI (US)

Correspondence Address: David A. Casimir, Esq., MEDLEN & CARROLL, LLP, Suite 350, 101 Howard Street, San Francisco, CA, 94105, US

(73) Assignees: The University of Michigan, Ann Arbor, MI; Paul R. Ervin, Jr., Ann Arbor, MI

(21) Appl. No.: 10/326,242

(22) Filed: Dec. 19, 2002

Related U.S. Application Data
(63) Continuation of application No. PCT/US00/16933, filed on Jun. 19, 2000.

(51) Int. Cl. C12Q 1/68; C07H 21/04; A61K 48/00; C12N 9/99

(52) U.S. Cl. 536/23.2; 514/44; 435/6; 435/184

(57) ABSTRACT

An Allelic varian of Mammastatin, MammC, nucleic acid sequence encoding the variant Mammastatin, and methods for breast cancer diagnosis and therapy using the variant sequence of the invention.

### FIELD OF THE INVENTION

This invention relates to mammary cell growth inhibitors useful in the diagnosis and treatment of breast cancer, and particularly to a variant sequence, MammC.

### BACKGROUND OF THE INVENTION

A novel, specific, mammary cell growth inhibitor, Mammastatin, has recently been identified and characterized. Mammastatin has been expressed from variant clones, MammA (PCT/US97/18026, SEQ ID NO: 1, ATCC# 97451, deposited Feb. 22, 1996) and MammB (PCT/US97/27147, SEQ ID NO: 2, ATCC# PTA-2091 deposited Jun. 15, 2000).

Mammastatin is produced and secreted by normal mammary cells, and is detected in blood samples of normal individuals. Blood concentrations of the mammary cell growth inhibitor, and particularly of the active, phosphorylated form of Mammastatin, are reduced or absent in breast cancer patients. Administration of protein comprising active Mammastatin (secreted from normal human breast cancer cells) is effective to reduce tumor size and number, and to prevent tumor growth in late stage cancer patients.

Mammastin is differentially expressed in mammary cells, being expressed in normal human mammary cells but expressed in reduced amount or not at all in cancerous breast tissues. Mammastat, is also detected in blood samples taken from normal individuals, but in reduced amount or not at all in the blood of patients with breast cancer.

### SUMMARY OF THE INVENTION

A variant nucleic acid sequence encoding Mammastatin has been identified, cloned, and sequenced (pMammC, SEQ ID NO: 3, ATCC# PTA-2090 deposited Jun. 15, 2000), as described in the Examples below. Like pMammA and pMammB, this variant clone can be used to diagnose breast cancer and/or to monitor cancer treatment. The new variant sequence also provides a useful therapeutic agent to inhibit mammary cell growth, prevent mammary tumor formation, and to prevent and/or treat breast cancer.

### BRIEF DESCRIPTION OF THE FIGURES

FIG. 1 is a computer scanned image of a Western blot showing pMammC expressed from a yeast vector and probed with anti-Mammastatin antibody, 7G6.

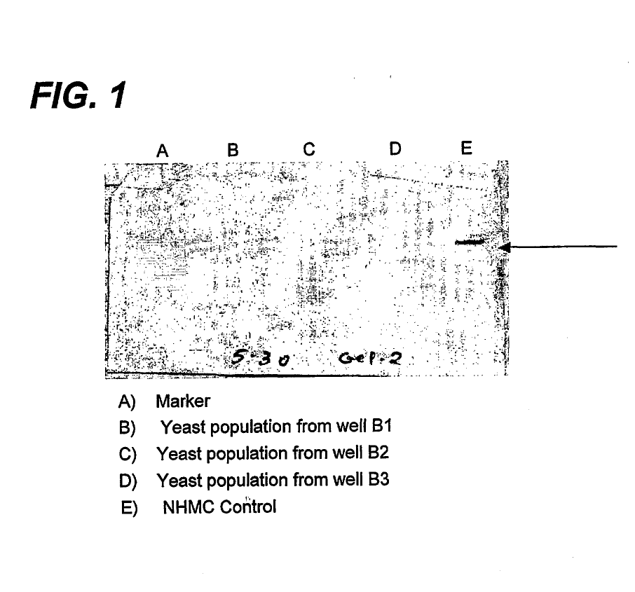

### DETAILED DESCRIPTION OF THE INVENTION

Proteins of the Invention:

“Mammastatin” is defined herein to mean mammary cell growth inhibitors produced by and active to inhibit the growth of human mammary cells. Active, inhibitory Mammastatin protein is reduced or absent in cancerous mammary cells. Mammastatin inhibitory activity is specific to mammary tissue, with little or no inhibitory activity in other tissue types.

Mammastatin is produced by normal, human mammary cells, and has previously been demonstrated be useful in the diagnosis and treatment of breast cancer (PCT/US97/18026). Two human Mammastatin clones (gammA and MammB) have been isolated and their sequences reported, as discussed above. MammC was discovered by subtraction hybridization screening of normal versus cancerous mammary cells, as described more fully below.

Like Mammastatin A and B, MammC appears, for example, in Western blots, as triplet bands, with one major band and one or two smaller, less prominent bands. This pattern of expression was demonstrated for Mammastatin A to be due to phosphorylation of the protein. Mammastatin has an approximate molecular weight of 53 kilodaltons when phosphorylated at two sites. Smaller sized Mammastatin forms, 49 and 44 kilodaltons, correspond to protein with reduced phosphorylation. Phosphorylation of the Mammastatin protein is correlated with its inhibitory activity.

Nucleic Acid Sequence

The nucleic acid sequence encoding Mammastatin C (MammC) shares significant sequence identity to nucleic acid sequences encoding Mammastatin A and B, and hybridizes to nucleic acid sequences enconding Mammastatin A and B under conditions of high stringency.

Nucleic acids encoding Mammastatin include those DNA inserts of MammA (PCT/US97/18026, ATCC# 97451, deposited Feb. 22, 1996); MammB 2 (PCT/US97/27147, ATCC# PTA-2091, deposited Jun. 15, 2000); and MammC of the invention, described herein (ATCC# PTA-2090, deposited Jun. 15, 2000).

Consensus sequences determined for the known Mammastatin DNA sequences are shown in the Comparative Sequence Table 1, below, and as SEQ ID NO: 1 (MammA); SEQ ID NO: 2 (amRB); SEQ ID NO: 3 (MammC).

Diagnostic Methods

The invention further provides an in vitro assay for detecting active, inhibitory Mammastatin in patient samples, including tissues, cells, and fluids. Breast cancer and advancing matastatic disease is diagnosed by correlating the presence and type of Mammastatin protein in a patient's sample with that of normal or cancerous mammary cells. A patient's blood or tissue sample is analyzed for Mammastatin protein, e.g., for the abundance of the protein and/or for its molecular weight forms. The absence or loss of Mammastatin protein, particularly of the higher molecular weight, phosphorylated forms, is correlated with advancing metastatic disease.

Analysis of Mammastatin can be performed using a variety of known analytical tools and methods, including immunoassays, hybridization, PCR techniques, and the like. Preferred are immunoassay, including ELISA, Western blot, and dot-blot analysis of a patient's sample methods, using anti-Mammastatin antibodies. Preferably, recombinant Mammastatin standards are used to provide a standard curve for reliable quantitation of inhibitor levels. Such immunoassays are exemplified by the dot-blot assays and Western blot assays shown in the examples -below. In an alternative preferred embodiment of the invention, tissue samples, such as tumor biopsies, are analyzed by immunohistochemistry, or by culturing a patient's tumor cells and examining the cultures for expression of Mammastatin.

In a particularly preferred embodiment, an assay for the diagnosis of breast cancer includes at least two specific antibodies: an antibody to identify the sampled tissue as epithelial tissue, such as an anti-cytokeratin antibody, and a specific anti-Mammastatn antibody. For example, using an immunoblot format, mammary tissue suspected of containing the cancer cells is homogenized, separated on an SDS/PAGE gel, transferred to membrane, and probed with both anti-keratin and anti-Mammastatin antibodies. Isotype specific second antibodies that are conjugated to a suitable marker system such as peroxidase or alkaline phosphatase are used to detect bound antibodies. Membranes containing bound first and second antibodies are then developed using known colormetric or fluorometric techniques and quantitated by known methods.

In the most preferred embodiment, the sample is analyzed for the size and/or phosphorylated forms of Mammastin, such as by Western Blot, using anti-Mammastatin antibodies. A decline or absence of the high molecular weight Mammastatin protein form correlates with advancing cancer.

Diagnostic kits of the invention include Mammastatin C protein or nucleic acid sequences encoding Mammastatin C, for example, as controls. Optionally, the diagnostic kit contains one or more antibodies that bind Mammastatin to be detected or quantified. Alternatively, the diagnostic kit includes one or more amplification primer or hybridization probe for the amplification and/or detection of nucleic acid sequences encoding MammC, for example, the primers used in the Examples below.

Therapeutic Use

Mammastatin for therapeutic use is produced from epithelial cell cultures under serum free conditions or by recombinant means. Preferably, Mammastatin protein in yeast or higher eucaryotic cells to achieve phosphorylation of the protein. Recombinant protein is produced in host cells or by synthetic means.

Functional Mammastatin is administered to patients by known method for the administration of phosphoprotein, preferably by injection, to increase inhibitor levels in the bloodstream and increase the inhibitor's interactions with the desired epithelial.

The protein may be delivered to the patient by methods known in the field for delivery of phosphorylated protein agents. In general, the inhibitor is mixed with the delivery vehicle and administered by injection.

The dosage of inhibitor to be administered may be determined by one skilled in the art, and will vary with the type of treatment modality and extent of disease. Since Mammastatin inhibits approximately 50% of mammary cancer cell growth at a concentration of 10 ng/ml and stops growth at about 20-25 ng/ml in vitro, a useful therapeutic dosage range is about 2.5 μg to about 250 μg administered daily dose. Preferred is approximately 125 μg daily administered dose. The aim of the administration is to result in a final body dose that is in the physiological (e.g. 15-50 ng/ml) or slightly higher range (for example, 25-75 ng/ml). For clinical use, the preferred dosage range is about 500 ng/ml for initial treatment of metastatic disease, followed by a maintenance dosage of about 50 ng/ml. In clinical studies using Mammastatin, an administered daily dose of about 50 ng/ml to about 750 ng/ml was sufficient to induce remission to Stage IV breast cancer patients.

Since active Mammastatin is a phosphortyated protein, it is anticipated that multiple doses of the inhibitor will be required to maintain growth inhibiting levels of Mammastatin in the patient's blood. Also, since Mammastatin generally acts as a cytostatic agent rather than a cytocidal agent, it is expected that a maximum effect of the inhibitor will require regular maintenance of inhibitor levels in breast cancer patients.

In its preferred use, Mammastatin is administered in high dosages (>50 ng/ml, preferably about 50-500 ng/ml) to induce tumor regression. Lower, maintenance doses (<50 ng/ml, preferably 20-50 ng/ml) are used to prevent cancer cell growth.

Clinical experience with administered Mammastatin in Stage IV breast cancer patients indicates a useful dose is that which maintains physiological levels of Mammastatin in the blood. Administration is preferably daily, but may be, for example, by continuous infusion, by slow release depot, or by injection once every 2-3 days. Anecdotal evidence suggests continuous administration may induce feedback inhibition, thus, a preferred administration scheme is to administer daily dose of Mammastatin for approximately 25-28 days, followed by 2-5 days without administration.

Diagnostic Assay

Assays of the present invention for detecting the presence of the functional inhibitor in human tissue and serum are useful in screening patients for breast cancer, for screening the population for those at high risk of developing breast cancer, for detecting early onset of breast cancer, and for monitoring patient levels of inhibitor during treatment. For example, analysis of a patient's blood Mammastatin, for example, may indicate a reduced amount of high molecular weight, phosphorylated Mammastatin, as compared with a normal control or with the patient's prior Mammastatin profile. Such a change is correlated with increased risk of breast cancer, with early onset of breast cancer, and with advancing metastatic breast cancer. Diagnostic assay for phosphorylated, active, approximately 53 kD Mammastatin preferably is by Western blot immunoassay, or ELISA using specific anti-Mammastatin antibodies. Screening, for example, in serum, is preferably by immunoassay, e.g., ELISA, Western blot, or dot blot assay.

For best results, the patient samples should be assayed within a short time of sampling (within one week), stored at 4PC (less than one year), or frozen for long term storage. Most preferably, samples are frozen until time of assay.

### EXAMPLES

The invention may be better understood by reference to the following Examples, which are not intended to limit the invention in any way.

### Example 1

### Subtraction Hybridization

Subtraction hybridization is a procedure for separating genes that are expressed differenctially in two different cell types. The theory is that two very similar cell types will express equivalent amounts of all genes/proteins when grown under similar conditions. Any genes that are expressed in excess should therefore be due to unique characteristics of a particular cell population.

To determe if a further gene for Mammastatin could be identified by subtraction hybridization, these studies were carried out. mRNA was isolated from normal human mammary cells obtained from surgery, and from MCF-7 breast cancer cells (ATCC). cDNA in equal amounts was made from each mRNA (5 ug) using reverse transcriptase. The cDNA was denatured and mixed with an excess (5×) of the other cell type mRNA. DNA:RNA hybrids were allowed to form. The double stranded DNA:RNA hybrids were passed over a hydroxyapitite column to bind double stranded nucleotides. The eluted cDNA was collected and subjected to second strand synthesis with DNA polymerase and random primers. Clones were produced by collecting the cDNA into bacterial plasmid vectors using blunt end ligation and specific DNA ends to create restriction sites for cloning into the plasmid. E coli was transformed with the vectors, and bacterial cultures grown out with the resultant recombinant DNA clones. Clones were isolated, and plasmid DNA inserts were sized and sequenced. The nucleic acid sequences obtained were compared with the known sequences for Mammastatin A and B.

Two clones were expressed in normal human mammary cells but not in breast cancer cells. One of these genes coded for a known calcium regulator, and the other, pMammC, encoded a further alleleic variant of the Mammastatin gene.

The nucleic acid sequence of pMammC was determined by dye terminator cycle sequencing using AmpliTaq and an ABI automated sequencing system. Products of the sequencing reaction are linearly amplified from small amounts of DNA template by thermal cycling of the annealing, extension, and denaturing steps of the reaction. Upon sequensing both strands of template DNA, a consensus sequence was determined for the mammastatin insert based on the raw sequensing data obtained. This consensus sequence of pMammC is shown in Table 1 below as compared with those of MammA and MammB.

TABLE 1
Comparison
MammA, MammB, MammC

| | |
|---|---|
| | 1 50 |
| pMamm A | (1) ------------------------------------------TGGGGCTC |
| pMamm B | (1) -------------------------------------------------- |
| pMamm C | (1) -------------------------------------------------- |
| | 51 100 |
| pMamm A | (9) CACCCCGGTGGCGGCCGCTCTAGAACTAGTGGATCCCCCGGGCTGCAGGA |
| pMamm B | (1) -------------------------------------------------- |
| pMamm C | (1) ------------------------------------------------GA |
| | 101 150 |
| pMamm A (59) ATTCGGCACGAGCACGGTGAAGAGACATGAGAGGTGTAGAATCCGTGGGA | |
| pMamm B | (1) ---CGGCACGAGCACGGTGAAGAGACATGAGAGGTGTAGAATAAGTGGGA |
| pMamm C | (3) ATTCGGCACGAGCACGGTGAAGAGACATGAGAGGTGTAGAATAAGTGGGA |
| | 151 200 |
| pMamm A | (109) GGCCCCCGGCGCCCCCCCGGTGTCCCCGCGACGGGCCCGGGGCGGGGTCC |
| pMamm B | (48) GGCCCCCGGCGCCCCCCCGGTGTCCCCGCGAGGGGCCCG----CGGGTCC |
| pMamm C | (53) GGCCCCCGGCGCCCCCCCGGTGTCCCCGCGAGGGGCCCGGGGCGGGGTCC |
| | 201 250 |
| pMamm A | (159) GCCGGCCCTGCGGGCCGCCGGTGAAATACCACTACTCTTATCGTTTTTTC |
| pMamm B | (94) GCCGGCCC-GCGGGC-GCCGGTGAAATACCACTACTCTGATCGTTTTTTC |
| pMamm C | (103) GCCGGCCCTGCGGGCCGCCGGTGAAATACCACTACTCTGATCGTTTTTTC |
| | 251 300 |
| pMamm A | (209) ACTGACCCGGTCGAGCGGCGGGGCGAGCCCCGAGGGGCTCTCGCTTCTGG |
| pMamm B | (142) ACTGACCCGGT-GAGGCGGGGGGCGAGCCCCGAGGGGCTCTCGCTTCTGG |
| pMamm C | (153) ACTGACCCGGTGAGGCGGGGGGGCGAGCCCCGAGGGGCTCTCGCTTCTGG |
| | 301 350 |
| pMamm A | (259) CGCCAAGCGCCCGGCCGCGCGCCGGCCGGGCGCGACCCGCTCCGGGGACA |
| pMamm B | (191) CGCCAAGCGCCCGGCCGCGCGCCGGCCGGGCGCGACCCGCTCCGGGGACA |
| pMamm C | (203) CGCCAAGCGCCCGGCCGCGCGCCGGCCGGGCGCGACCCGCTCCGGGGACA |
| | 351 400 |
| pMamm A | (309) GTGCCAGGTGGGGAGTTTGACTGGGGCGGTACACCTGTCAAACGGTAACG |
| pMamm B | (241) GTGCCAG-TGGGGAGTTTGACTGGGGCGGTACACCTGTCAAACGGTAACG |
| pMamm C | (253) GTGCCAGGTGGGGAGTTTGACTGGGGCGGTACACCTGTCAAACGGTAACG |
| | 401 450 |
| pMamm A | (359) CAGGTGTCCTAAGGCGAGCTCAGGGAGGACAGAAACCTCCCGTGGAGCAG |
| pMamm B | (290) CAGGTGTCCTAAGGCGAGCTCAGGGAGGACA-AAACCTCCCGTGGAGCAG |
| pMamm C | (303) CAGGTGTCCTAAGGCGAGCTCAGGGAGGACAGAAACCTCCCGTGGAGCAG |
| | 451 500 |
| pMamm A | (409) AAGGGCAAAAGCTCGCTTGATCTTGATTTTCAGTACGAATACAGACCGTG |
| pMamm B | (339) AAGGGCAAAA-------TGATCTTGATTTTCAGTACGAATACAGACCGTG |
| pMamm C | (353) AAGGGCAAAAGCTCGCTTGATCTTGATTTTCAGTACGAATACAGACCGTG |
| | 501 550 |
| pMamm A | (459) TAAGCGGGGCCTCACGATCCTTCTGACCTTTTGGGTTTTAAGCAGGAGGT |
| pMamm B | (382) AAAGCGGGGCCTCA-GATC-TTCTGACCTTTTGGGTTTTAAGCAGGAGGT |
| pMamm C | (403) AAAGCGGGGCCTCACGATCCTTCTGACCTTTTGGGTTTTAAGCAGGAGGT |
| | 551 600 |
| pMamm A | (509) GTCAGAAAAGTTACCACAGGGATAACTGGCTTGTGGCGGCCAAGCGTTCA |
| pMamm B | (430) GTCAGAAAAGTTACCACAGGGATAACTGGCTTGTGGCGGCCAAGCGTTCA |
| pMamm C | (453) GTCAGAAAAGTTACCACAGGGATAACTGGCTTGTGGCGGCCAAGCGTTCA |
| | 601 650 |
| pMamm A | (559) TTAGGACGTCGCTTTTTGATCCTTCGATGTCGGCTCTTCCTATCATTGTG |
| pMamm B | (480) AAGCGACGTCGCTTTTTGATCCTTCGATGTCGGCTCTTCCTATCATTGGG |
| pMamm C | (503) TAGCGACGTCGCTTTTTGATCCTTCGATGTCGGCTCTTCCTATCATTGTG |
| | 651 700 |
| pMamm A | (609) TAGCAGAATTCACCAAGCGTTGGATTGTTCACCCACTAATAGGGAACGTG |
| pMamm B | (530) AAGCAGAATTCACCAAGCGTTGGATTGTTCACCCACTAATAGGGAACGTG |
| pMamm C | (553) AAGCAGAATTCACCAAGCGTTGGATTGTTCACCCACTAATAGGGAACGTG |
| | 701 750 |
| pHamm A | (659) AGCTGGGTTTAGACCGTCGTGAGACAGGTTATTTTTACCCTACTGATGAT |
| pMamm B | (580) AGCTGGGTTTAGACCGTCGTGAGACAGGTT-TGTTTACCCTACTGATGAT |
| pMamm C | (603) AGCTGGGTTTAGACCGTCGTGAGACAGGTTAGTTTTACCCTACTGATGAT |
| | 751 800 |
| pMamm A | (709) TGTTTGTTGCCATGGTTATCCTGCTCAGTACGAGAGGAACCGCAGGTTCA |
| pMamm B | (629) GTGTTGTTGCCATGGTAATCCTGCTCAGTACGAGAGGAACCGCAGGTTCA |
| pMamm C | (653) GTGTTGTTGCCATGGTAATCCTGCTCAGTACGAGAGGAACCGCAGGTTCA |
| | 801 850 |
| pMamm A | (759) GACATTTGGTGTATGTGCTTGGCTGAGGAGCCAATGGGGCGAAGCTACCA |
| pMamm B | (679) GACATTTGGTGTATGTGCTTGGCTGGGGAGCCAATGGGGCGAAGCTACCA |
| pMamm C | (703) GACATTTGGTGTATGTGCTTGGCTGAGGAGCCAATGGGGCGAAGCTACCA |
| | 851 900 |
| pMamm A | (809) TCTGTGGGATTATGACTGA-CGC-TCTAAGTCATGAATCCCGCCCAGGCG |
| pMamm B | (729) TCTGTGGGATTATTACTGAACGCCTCTAAGTCA-GAATCCCGCCCAGGCG |
| pMamm C | (753) TCTGTGGGATTATGACTGAACGCCTCTAAGTCA-GAATCCCGCCCAGGCG |
| | 901 950 |
| pHamm A | (857) GAACGATACGGCAGCGCCGCGGAGCCTCGCTTGGCCTCGGATTAGCCGGT |
| pMamm B | (778) GAACGATACGGCAGCGCCGCGGAGCCTCGGTTGGCCTCGGATG-GCCGGT |
| pManm C | (802) GAACGATACGGCAGCGCCGCGGAGCCTCGGTTGGCCTCGGATA-GCCGGT |
| | 951 1000 |
| pMamm A | (907) CCCCCGCCTGTCCCCGCCGGCGGGCCGCCCCCCCCCCTCCACGCGCCCCG |
| pMamm B | (827) CCCCCGCCTGTCCCCGCCGGCGGGC-GCCCCCCCCCCTCCACGCGCCCCG |
| pMamm C | (851) CCCCCGCCTGTCCCCGCCGGCGGGCCGCCCCCCCCCCTCCACGCGCCCCG |
| | 1001 1050 |
| pMamm A | (957) CGCGCGCGGGAGGGCGCGTGCCCCGCCGCGCGCCGGGACCGGGGTCCGGT |
| pMamm B | (876) CGCGCGCGGGAGGGCGCGTGCCCCGCCGCGCGCCGGGACCGGGGTCCGGT |
| pMamm C | (901) CGCGCGCGGGAGGGCGCGTGCCCCGCCGCGCGCCGGGACCGGGGTCCGGT |
| | 1051 1100 |
| pMamm A | (1007) GCGGAGTGCCCTTCGTCCTGGGAAACGGGGCGCGGCCGGAAAGGCGGCCG |
| pMamm B | (926) GCGGAGTGCCCTTCGTCCTGGGAAACGGGGCGCGGCCGGAAAGGCGGCCG |
| pMamm C | (951) GCGGAGTGCCCTTCGTCCTGGGAAACGGGGCGCGGCCGGAAAGGCGGCCG |
| | 1101 1150 |
| pMamm A | (1057) CCCCCTCGCCCGTCACGCACCGCACGTTCGTGCT---CGTGCCGAATTCG |
| pMamm B | (976) CCCCCTCGCCCGTCACGCACCGCACGTTCGTGCT---CGTGCCGAATTCG |
| pMamm C | (1001) CCCCCTCGCCCGTCACGCACCGCACGTTCGTGCT---CGTGCCGAATTCG |
| | 1151 1200 |
| pMamm A | (1104) GCACGAGTGCACCCATTCACAATATACATACAAGTGCATGTATCTTTATG |
| pMamm B | (1023) GCACGAGTAGCACCATTCACAATAGACATACAAGTGCATGTATCTTTATT |
| pMamm C | (1048) GCACGAGTAGCACCATTCACAATAGACATACAAGTGCATGTATCTTTATG |
| | 1201 1250 |
| pMamm A | (1154) ATATAATGAATTCTTTTCCTTTGGGTAGATATCCAGTAGTGGGATTGCTA |
| pMamm B | (1073) ATATAATGAATTCTTTTCCTTTGGGGAGATATCCAGTAGTGGGATTGCTA |
| pMamm C | (1098) ATATAATGAATTCTTTTCCTTTGGGTAGATATCCAGTAGTGGGATTGCTA |
| pMamm A | (1204) GATCACCTGGTAGTTCTATTTCTGGTTTATTTAGAAATCTTCATACTGAT |
| pMamm B | (1123) GATCACCTGGTAGTTCTATTTCTGGTTTATTGAGAAATCTTCATACTGAT |
| pMamm C | (1148) GATCACCTGGTAGTTCTATTTCTGGTTTATTGAGAAATCTTCATACTGAT |
| | 1301 1350 |
| pMamm A | (1254) TTCCATAGAGGTTGTACAAATTTACATCCCTACCAAAGTGATTTTTTTAA |
| pMamm B | (1173) TTCCATAGAGGTTGTACAAATTTACATCCCTACCAA-GTGATTTTTTTAA |
| pMamm C | (1198) TTCCATAGAGGTTGTACAAATTTACATCCCTACCAA-GTGATTTTTTTAA |
| | 1351 1400 |
| pMamm A | (1304) ATATGAAAGAATGGTCTGGAGAAATGCCCCTCATTAGTATCCCCCTTTTA |
| pMamm B | (1222) ATATGAAAGAATGGTCTGGAGAAATGCCCCTCATTAGTATCCCCCTTTTA |
| pMamm C | (1247) ATATGAAAGAATGGTCTGGAGAAATGCCCCTCATTAGTATCCCCCTTTTA |
| | 1401 1450 |
| pMamm A | (1354) CCTCTCTACTGCAGAATGACTTCAAGGGGTACAGGTATTTACAAGTTTCA |
| pMamm B | (1272) CCTCTCTACTGCAGAATGACTTCAAGGGGTACAGGTATTTACAAGTTTCA |
| pMamm C | (1297) CCTCTCTACTGCAGAATGACTTCAAGGGGTACAGGTATTTACAAGTTTCA |
| | 1451 1500 |
| pMamm A | (1404) TTATACAGACAAATTGAATATTGAAATTTTCTGCATAAGAGGCACAGATT |
| pMamm B | (1322) TTATACAGACAAATTGAATATTGAAATTT-CTGCATTAGAGGCACAGATT |
| pMamm C | (1347) TTATACAGACAAATTGAATATTGAAATTT-CTGCATAAGAGGCACAGATT |
| | 1501 1550 |
| pMamm A | (1454) TTAGGATTCAAAGTTGTATGAACAAGGACAAGTGCTCTAGGGACTTGCAA |
| pMamm B | (1371) TTAGGATTCAAAGTTGTAAGAACAAGGACAAGTGCTCTAGGGACTTGCAA |
| pMamm C | (1396) TTAGGATTCAAACTTGTATGAACAAGGACAAGTGCTCTAGGGACTTGCAA |
| | 1551 1600 |
| pMamm A | (1504) AGCTGGAATTGGAAATCTCAGATGAAATACATTTCTAGTAGTACCACCAG |
| pMamm B | (1421) AGCTGGAATTGGAAATCTCAGAAGAAATACATTTCTAGTAGTACCACCAG |
| pMamm C | (1446) AGCTGGAATTGGAAATCTCAGATGAAATACATTTCTAGTAGTACCACCAG |
| | 1601 1650 |
| pMamm A | (1554) CATATATTCTACTGAATTGGCTTTTGTGATCATCATTAATACCTACTTAT |
| pMamm B | (1471) CATATATTCTACTGAATTGGCTTT-GTGATCATCATTTATACCTACTTAT |
| pMamm C | (1496) CATATATTCTACTGAATTGGCTTT-GTGATCATCATTAATACCTACTTAT |
| | 1651 1700 |
| pMamm A | (1604) TAAAACTAATGAAAAGGGTTTATATCAAATATACTTTAAGGTATAAAAAT |
| pMamm B | (1520) TAAAACTAATGAAAAGGGTTTATATCAAATATACTTTAAGGTAAAAAAAT |
| pMamm C | (1545) TAAAACTAATGAAAAGGGTTTATATCAAATATACTTTAAGGTATAAAAAT |
| | 1701 1750 |
| pMamm A | (1654) CAAATTATAGGTAAAGCTGTTTTCTTTAGCATTTTAATTTCAAAACATAA |
| pMamm B | (1570) CAAATTATAGGAAAAGCTGTTTTCTTTTGCATTTTAATTTCAAAACAAAA |
| pMamm C | (1595) CAAATTATAGGTAAAGCTGTTTTCTTTAGCATTTTAATTTCAAAACATAA |
| | 1751 1800 |
| pMamm A | (1704) AATAGCTACCGTCTATTGGGCAT--TTATA-CTGTACGAGACACTGTGTT |
| pMamm B | (1620) AATAGCTACCGTCTATTGGGCAT--TTATA-CTGTACCAGACACTGTGTT |
| pMamm C | (1645) AATAGCTACCGTCTATTGGGCAT--TTATA-CTGTACCAGACACTGTGTT |
| | 1801 1850 |
| pMamm A | (1751) TGTCACATTTCAAAAATGTTCTCATGGTAATGTTCACAATAATTCTGTCG |
| pMamm B | (1667) TGTCACATTTCAAAAATGTTCTCATGGTAATGTTCACAATAATTCTGTAG |
| pMamm C | (1692) TGTCACATTTCAAAAATGTTCTCATGGTAATGTTCACAATAATTCTGTAG |
| | 1851 1900 |
| pMamm A | (1801) GGTGAGAAAATAGTCTTACCGTAGTAAGACTATTCAGTAAAACGAAACCT |
| pMamm B | (1717) GGTGGAGAAATAGTCTTACCGTAGTAAGACTAATTCAG-AAACGAAACCT |
| pMamm C | (1742) GGTGAG-AAATAGTCTTACCGTAGTAAGACTATTCAGT-AAACGAAACCT |
| | 1901 1950 |
| pMamm A | (1851) CTGAACCTTGGAGTTCAACTTGCGCAAAGTTAGTAACAGGACTACGACTT |
| pMamm B | (1765) CTGAACCTTGGAGTTCAACTTGCGCAAAGTTAGTAACAGGACTAGGACTT |
| pMamm C | (1790) CTGAACCTTGGAGTTCAACTTGCGCAAAGTTAGTAACAGGACTAGGACTT |
| | 1951 2000 |
| pMamm A | (1901) GAA--CCTGAACCATCACACTCGAGAT--CTCT---CCATACCACACTGC |
| pMamm B | (1815) GAA--CCTGAACCATCACACTCCAGAT--CTCT---CCATACCACACTGC |
| pMamm C | (1840) GAA--CCTGAACCATCACACTCCAGAT--CTCT---CCATACCACACTGC |
| | 2001 2050 |
| pMamm A | (1944) TAGCACATG---TGCCTGT---CATCTTATTCCTGGCTCC---------- |
| pMamm B | (1858) TAGCACATG---TGCCTGT---CATCTTATTCCTGGCTCC---------- |
| pMamm C | (1883) TAGCACATG---TGCCTGT---CATCTTATTCCTGGCTCCTGTTATT-TC |
| | 2051 2100 |
| pMamm A | (1978) CTTTTTTATTTCCTTTCCCTT--CCTCCCACAACCCCTTTTTCCCCCC-- |
| pMamm B | (1892) CTKYTT-ATTTCCTTTCCCTT--CCTCCCACAACCCCTTTTTCCCCCC-- |
| pMamm C | (1926) CCTTTTTATTTCCTTTCCCTT--CCTCCCACAACCCCTTTTTCCCCCC-- |
| | 2101 2150 |
| pMamm A | (2024) -ATTTCTTT-CTTTCTTTTTATTTGTTAATTACATAACTAATACATGTTT |
| pMamm B | (1937) -ATTTCTTTTCTTTCTTTTTATTTGTTAATTACATAACTAATACATGTTT |
| pMamm C | (1972) -ATTTCTTTTCTTTCTTTTTAATTGTTAATTACATAACTAATACATGCTT |
| | 2151 2200 |
| pMamm A | (2072) ATGAGAACAATTGATATAGCACAAAAGGATATAAAGTACGGGGGAGTGAT |
| pMamm B | (1986) ATCAGAACAATTGATATAGCACAAAAGGATATAAAGTACGGGTGAGTGAT |
| pMamm C | (2021) ATCAGAACAATTGATATAGCACAAAAGGATATAAAGTACGGGTGAGTGAT |
| | 2201 2250 |
| pMamm A | (2122) AGCTCATCCCTGTAATCCTAGCACTTTGGAAGGCCAAGGCAG-GCAGATC |
| pMamm B | (2036) AGCTCATCCCTGTAATC-TAGCACTTTCGAAGGCCAAGGCAG-GCAGATC |
| pMamm C | (2071) AGCTCATCCCTGTAATCCTAGCACTTTGGAAGGCCAAGGCAG-GCAGATC |
| | 2251 2300 |
| pMamm A | (2171) ACTTTGAGTCCAGAGTTCGAGACCAGCCTGGGCAACATGGTGAAACCCTG |
| pMamm B | (2084) ACTT-GA-TCCAGAGTTCGAGACCAGCCTGGGCAACATGGTGAAACCCTG |
| pMamm C | (2120) ACTT-GAGTCCAGAGTTCGAGACCAGCCTGGGCAACATGGTGAAACCCTG |
| | 2301 2350 |
| pMamm A | (2221) TCTCTACAAAAAAATACAAAAAA-TTTAGCCGGGCGTGCTGGCACAGACC |
| pMamm B | (2132) TCTCTACAAAAAAATACAAAAA--TTTAGCCGGGCGTGCTGGCACACACC |
| pMamm C | (2169) TCTCTACAAAAAAATACAAAAA--TTTAGCCGGGCGTGCTGGCACACACC |
| | 2351 2400 |
| pMamm A | (2270) TGTAGTCTCAGCTACTCTGAGGGCTGAGGTGGGAAGATTGATTGAGCCCA |
| pMamm B | (2180) TGTAGTCTCAGCTACTCTGAGGGCTGAGGTGGGAAGATTGATTGAGCCCA |
| pMamm C | (2217) TGTAGTCTCAGCTACTCTGAGGGCTGAGGTGGGAAGATTGATTGAGCCCA |
| | 2401 2450 |
| pMamm A | (2320) GGAGGTGGAAGCTGCAGCAGTGCGCTGAGATTGCGCCATTGCACTCCAGC |
| pMamm B | (2230) GGAGGTGGAAGCTGCAGCAGTGCGCTGAGATTGCGCCATTGCACTCCAGC |
| pMamm C | (2267) GGAGGTGGAAGCTGCAGCAGTGCGCTGAGATTGCGCCATTGCACTCCAGC |
| | 2451 2500 |
| pMamm A | (2370) CTGGGTGAGAGAGAGAGACCCTGTCTCCAAAAAAAAAAAAAAAAAAAAA- |
| pMamm B | (2280) CTGGGTGAGAGAGAGAGACCCTGTCTTCAAAAAAAAAAAAAAAAAAA--- |
| pMamm C | (2317) CTGGGTGAGAGAGAGAGACCCTGTCTCAAAAAAAAAAAA----------- |
| | 2501 2532 |
| pMamm A | (2419) -------------------------------- |
| pMamm B | (2327) -------------------------------- |
| pMamm C | (2356) -------------------------------- |

### Example 2

### Expression and Inhibitory Activity

pMammC was used as a DNA source to create new yeast expression vectors. MammnC cDNA was digested with BamHI/xbaI and the cDNA insert was isolated. The PiCZ yeast shuttle vector was digested with BamHI and xbaI, the vector purified, and ligated with the MammC cDNA insert. The ligation mix BW LB (low salt) Agar and Zeocin plates were transformed, using RecA cells. Positive candidates were selected through PCR and miniprep plasmid isolation and digestion. The plasmid DNA was then purified from the right clones (PicZx-Mam).

To integrate the DNA into yeast, the PicZx-Mam plasmid was linearized with single cutter enzyme Bstx-l to allow efficient gene intergration into the pichia genome. PicZ vectors do not contain an origin of replication, so only the recombinants will grow under the selection of the antibiotic Zeocin. Gs115 yeast strain was used to isolate the yeast competent cells. Yeast competent celss and linearlized PicZx-Mam plasmid DNA were used for the transformation. After 4 hours of incubation at 31° C. the mix was spread at different dilutions on Yepp+Agar+Zeocin plates, and incubated for 4 days at 31° C. Single yeast colonies were isolated by streaking onto fresh plates.

Three individual yeast colonies were picked and transferred to separate liquid growth medias and grown for father plating. Liquid cultures were again spread onto yeast plates. Five colonies were picked from each plate and grown in suspension culture for analysis. Initial screening was perfomed on culture supernatants by dot blot with the anti-Mammastatin antibody 7G6. Yeast cultures that demonstrated an enhanced signal on Dot blot were selected for further analysis. Cells and growth media (supernatant) were separated by centrifugation and analyzed by Western Blot using the 7G6 anti-Mammastatin monoclonal antibody. Yeast cultures that were positive by Western blot were also tested for growth inhibitory activity on MCF-7 breast cancer cells.

Growth assays were performed by plating MCF-7 at low density (10⁴ cells/ml) in 12 well plates, one millimeter per well in MEM growth media with 10% FBS supplement. Cells were allowed to attach overnight and were then treated with either yeast growth media, yeast culture supernatant, or yeast cell pellet extract as a 10% (v/v) supplement. Yeast pellet extract was produced by repeated freeze thawing of cell extracts in buffer containing 0.5% Triton X-100. MCF-7 cell cultures were allowed to grow for six days before counting.

| Treatment Sample | Mean cell number | % Inhibition | % Error |
|---|---|---|---|
| Control | 6866 | 0 | 11 |
| BLac Z Pellet | 4390 | 36 | 4 |
| B3 Supernatant | 1911 | 72 | 29 |
| B3 Pellet | 1456 | 79 | 2 |
| C2C3 Mix Supernatant | 3063 | 55 | 9 |
| B2 Pellet | 877 | 87 | 5 |
| B1 Supernatant | 24946 | — | 7 |
| B1 Pellet | 1506 | 78 | 7 |
| A5 Supernatant | 28569 | — | — |
| A5 Pellet | 2405 | 65 | 19 |
| A4 Supernatant | 25852 | — | — |
| A4 Pellet | 2048 | 72 | 12 |
| A3 Supernatant | 22097 | — | — |
| A3 Pellet | 1830 | 73 | 4 |
| A1 Supernatant | 17186 | — | — |
| A1 Pellet | 2161 | 69 | 12 |

Although there was some inhibitory activity caused by the Lac Z pellet there was significantly more inhibition from the pellets produced by the positive clones. In addition, one supernatant, the mixtures of C2 and C3 supernatants had inhibitory activity while the majority of the supernatants were positive. This suggests that there is Mammastatin induced inhibitory activity that is largely confined to the cell pellet from these cultures.

We claim:

1. A nucleic acid sequence encoding Mammastatin having the sequence of Seq ID NO: 3.

2. A diagnostic assay for the detection of breast cancer comprising a nucleic acid sequence of Seq ID No: 3.

3. A therapeutic composition comprising a nucleic acid sequence of Seq ID No: 3.

4. A method for the treatment of breast cancer comprising administering to a patient a therapeutically effective amount of Mammastatin produced by expression of Seq ID No: 3.

5. Mammastatin variant C encoded by Seq ID NO: 3.
